(19) United States
(12) Patent Application Publication
(10) Pub. No.: US 2025/0017589 A1
(43) Pub. Date: Jan. 16, 2025

(54) LEFT ATRIAL APPENDAGE OCCLUSION

(71) Applicant: Medtronic, Inc., Minneapolis, MN (US)

(72) Inventor: Sebastian TEODORU, Santa Ana, CA (US)

(73) Assignee: Medtronic, Inc., Minneapolis, MN (US)

(21) Appl. No.: 18/615,099

(22) Filed: Mar. 25, 2024

Related U.S. Application Data
(60) Provisional application No. 63/512,957, filed on Jul. 11, 2023.

(51) Int. Cl. A61B 17/12; A61B 17/00

(52) U.S. Cl. CPC A61B 17/12122; A61B 17/0057; A61B 2017/00632

(57) ABSTRACT

The present application relates to an occlusion device for occluding a left atrial appendage. The device has a stem with an elongate tubular body and a central lumen that extends from a proximal end to a distal end with a proximal portion arranged at the proximal end of the stem, The proximal portion is arranged to engage with an opening of the left atrial appendage. The device includes an expandable member arranged at the distal end of the stem. The central lumen of the stem is in fluid communication with the expandable member and the expandable member is arranged to be positioned inside a left atrial appendage. The expandable member is operable to at least partially expand and displace the proximal portion in a direction towards the expandable member.

### CROSS-REFERENCE TO RELATED APPLICATIONS

This application claims the benefit of U.S. Provisional Patent Application Ser. No. 63/512,957, filed Jul. 11, 2023, the entire content of which is incorporated herein by reference.

### FIELD OF THE INVENTION

The present invention relates to a method of occluding a left atrial appendage and an occlusion device for occluding a left atrial appendage.

### BACKGROUND OF THE INVENTION

The human heart is a four chambered, muscular organ that provides blood circulation through the body during a cardiac cycle. The four main chambers include the right atrium RA and right ventricle RV which supplies the pulmonary circulation, and the left atrium LA and left ventricle LV which supplies oxygenated blood received from the lungs into systemic circulation. To ensure that blood flows in one direction through the heart, atrioventricular valves (tricuspid valves TV and mitral valves MV) are present between the junctions of the atria and the ventricles, and semi-lunar valves (pulmonary valve and aortic valve) govern the exits of the ventricles leading to the lungs and the rest of the body.

The heart also includes a left atrial appendage LAA, which is a small, car-shaped sac in the muscle wall of the left atrium LA. In normal hearts, when the heart contracts, the blood in the left atrium LA and the left atrial appendage LAA is squeezed out of the left atrium LA and into the left ventricle LV. The LAA has minimal influence on cardiac output and is generally considered to be non-functional structure in the heart.

In atrial fibrillation, an irregular heartbeat causes blood flow to slow enabling clots to form. Because the left atrial appendage LAA is a small sac or pouch, blood may collect there and form clots. The clots often cause thromboembolic complications and risk to the patient. This risk increases if the thrombus or fragments of the thrombus dislodge. If all or a portion of the thrombus flows downstream, it is highly likely that the free material will become trapped in smaller and more tortuous anatomy. This increases a patient's risk for cerebral stroke or peripheral embolism. Thus, in some cases, it may be desirable to exclude or occlude the left atrial appendage LAA such that clots do not form in the left atrial appendage LAA, and if they do, they cannot escape the left atrial appendage LAA. While medications can be taken to control blood clotting and decrease the chance of stroke, a proportion of patients cannot take such medications or follow the strict regimen necessary for continued protection.

To reduce the above-mentioned risks, it is desirable to close the left-atrial appendage to reduce the occurrence of thrombus formation and the risk of thromboembolism. The present disclosure relates to improvements in catheter-based occlusion systems for occluding or excluding the left atrial appendage.

### SUMMARY OF THE INVENTION

A first aspect of the invention provides an occlusion device for occluding a left atrial appendage, the device comprising: a stem with an elongate tubular body and a central lumen that extends from a proximal end to a distal end, a proximal portion arranged at the proximal end of the stem; wherein the proximal portion is arranged to engage with an opening of the left atrial appendage, an expandable member arranged at the distal end of the stem; wherein the central lumen of the stem is in fluid communication with the expandable member and wherein the expandable member is arranged to be positioned inside a left atrial appendage; and wherein the expandable member is operable to at least partially expand and displace the proximal portion in a direction towards the expandable member.

Optionally, the expandable member is arranged to receive fluid through the central lumen of the stem so as to at least partially expand within the left atrial appendage.

Optionally, the expandable member is asymmetric, having a width greater than its height.

Optionally, the width of the expandable member is substantially oval or elliptical shaped.

Optionally, the expandable member comprises a nitinol mesh.

Optionally, the distal end of the stem extends through the expandable member and is secured to an outer surface at a distal end of the expandable member.

Optionally, the stem is in fluid communication with the expandable member through at least one aperture extending from the central lumen of the stem.

Optionally, the aperture extends perpendicularly from the central lumen.

Optionally, the stem is arranged to be positioned in a distal end of a catheter so that the distal end of the catheter extends around the stem, wherein the stem is moveable between a first position and a second position, wherein in the first position, the at least one aperture is occluded and no longer in fluid communication with the expandable member, and in a second position, the at least one aperture is in fluid communication with the expandable member.

Optionally, the stem further comprises at least one aperture at the distal end of the stem, wherein the at least one aperture is arranged to be in fluid communication with the left atrial appendage.

Optionally, the expandable member comprises at least two chambers.

Optionally, the stem is in fluid communication with each chamber through at least one respective aperture so that each chamber is arranged to be expanded separately.

Optionally, the proximal portion is dome shaped and an apex of the dome is proximal to the expandable member.

Optionally, the proximal portion comprises a bioinert polymer.

Optionally, the proximal portion is at least partially coated with a material coating.

A further aspect of the invention provides an occlusion assembly for occluding a left atrial appendage, the assembly comprising: a catheter with an elongate tubular body and a central lumen that extends from a proximal end to a distal end of the catheter, the distal end arranged to be positioned at an opening of the left atrial appendage; an occlusion device comprising a stem with an elongate tubular body and a central lumen that extends from a proximal end to a distal end, a proximal portion arranged at the proximal end of the stem; wherein the proximal portion is arranged to engage with an opening of the left atrial appendage,
an expandable member arranged at the distal end of the stem; wherein the central lumen of the stem is in fluid communication with the expandable member and the expandable member is arranged to be positioned inside a left atrial appendage; and wherein the expandable member is operable to at least partially expand and displace the proximal portion in a direction towards the expandable member, wherein the occlusion device is releasably connected to the distal end of the catheter.

Optionally, the occlusion assembly further comprises a fluid source coupled to the catheter, wherein the fluid source is operable to deliver fluid to the expandable member.

Optionally, when the stem of the occlusion device further comprises at least one aperture at the distal end of the stem and wherein the at least one aperture is arranged to be in fluid communication with the left atrial appendage, and wherein the fluid source is further operable to deliver fluid into the left atrial appendage.

Optionally, the occlusion assembly further comprises a plunger with a proximal end and a distal end,
wherein the proximal end of the plunger is housed in the distal end of the catheter and the distal end of the plunger is releasably connected to the proximal portion of the occlusion device, so that the occlusion device is releasably connected to the catheter through the plunger.

Optionally, the fluid is biogel.

Optionally, the biogel comprises radiopaque markers.

A further aspect of the invention provides a method of occluding a left atrial appendage, the method comprising: providing an occlusion assembly for occluding a left atrial appendage, the assembly comprising: a catheter with an elongate tubular body and a central lumen that extends from a proximal end to a distal end of the catheter, the distal end arranged to be positioned at an opening of the left atrial appendage; an occlusion device comprising a stem with an elongate tubular body and a central lumen that extends from a proximal end to a distal end, a proximal portion arranged at the proximal end of the stem; wherein the proximal portion is arranged to engage with an opening of the left atrial appendage, an expandable member arranged at the distal end of the stem; wherein the central lumen of the stem is in fluid communication with the expandable member and the expandable member is arranged to be positioned inside a left atrial appendage; and wherein the expandable member is operable to at least partially expand and displace the proximal portion in a direction towards the expandable member, wherein the occlusion device is releasably connected to the distal end of the catheter, the method comprising: positioning the distal end of the catheter with the occlusion device next to an opening of the left atrial appendage; advancing the expandable distal portion of the occlusion device into the left atrial appendage, and at least partially expanding the expandable member so as to move the proximal portion into sealing engagement with the opening of the left atrial appendage.

Optionally, the occlusion device is detached from the catheter after the proximal portion has created a seal against the opening of the left atrial appendage.

Optionally, when the occlusion assembly further comprises a fluid source coupled to the catheter such that the fluid source is operable to deliver fluid to the expandable member, wherein at least partially expanding the expandable member comprises delivering fluid into the expandable member.

Optionally, the stem is in fluid communication with the expandable member through at least one aperture extending from the central lumen of the stem, and wherein delivering fluid into the expandable member comprises:
delivering fluid through the stem of the occlusion device and through the at least one aperture extending from the central lumen of the stem and into the expandable member.

Optionally, when the stem of the occlusion device further comprises at least one aperture at the distal end of the stem and wherein the at least one aperture is arranged to be in fluid communication with the left atrial appendage, the method further comprises:
delivering fluid through the stem of the occlusion device through the at least one aperture at the distal end of the stem and into the left atrial appendage.

Optionally, the stem is further comprises a catheter, and wherein the stem is positioned in the distal end of a catheter so that the distal end of the catheter extends around the stem, and wherein the stem is moveable between a first position and a second position, wherein in the first position, the at least one aperture is occluded and no longer in fluid communication with the expandable member, and in a second position, the at least one aperture is in fluid communication with the expandable member, the method further comprises:
moving the stem into the second position before fluid is delivered to the expandable member.

Optionally, the method further comprises moving the stem into the first position after fluid is delivered to the expandable member; and delivering fluid through the at least one aperture at the distal end of the stem and into the left atrial appendage.

Optionally, the fluid is a biogel, and the method further comprises incorporating radiopaque markers into the biogel.

### BRIEF DESCRIPTION OF THE DRAWINGS

Embodiments of the invention will now be described with reference to the accompanying drawings, in which:

FIG. 1 is a schematic sectional illustration of a heart;

FIG. 2 illustrates a diagrammatic view of a patient undergoing a cardiovascular procedure to occlude a left atrial appendage;

FIG. 3 illustrates an exemplary occlusion assembly;

FIG. 4 illustrates an exemplary left atrial appendage;

FIG. 5 illustrates an exemplary occlusion assembly with the occlusion device in an expanded state;

FIGS. 6A-6D are schematic views of a part of an occlusion device according to an embodiment;

FIG. 7A illustrates a distal end of a catheter adjacent to a the left atrial appendage;

FIG. 7B illustrates a distal end of the catheter inserting into the left atrial appendage;

FIG. 7C illustrates the occlusion device in an expanded state in the left atrial appendage;

FIG. 7D illustrates the occlusion device with an expanded expandable member in the left atrial appendage;

FIG. 7E illustrates the catheter detached from the occlusion device;

FIGS. 8A and 8B illustrate cross-sectional views of different aperture arrangements of the occlusion device;

FIGS. 9A and 9B illustrate another embodiment of an exemplary occlusion assembly

FIG. 10 illustrates an exemplary method of occluding a left atrial appendage.

### DETAILED DESCRIPTION OF EMBODIMENT(S)

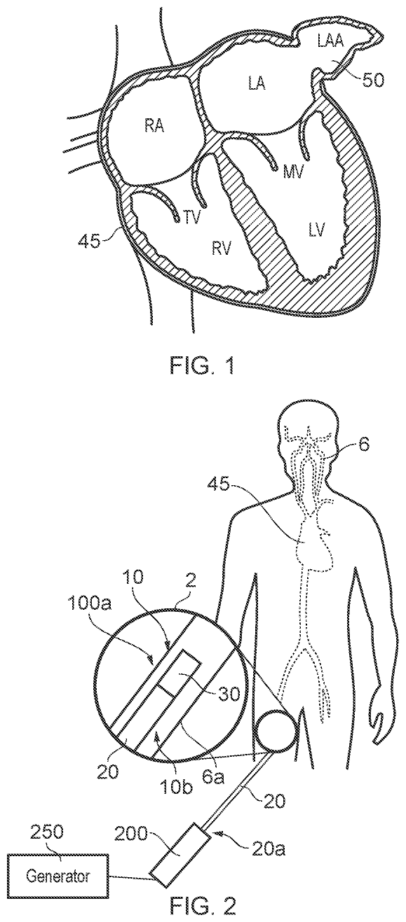

FIG. 1 is a schematic sectional illustration of a human heart 45 that depicts the four heart chambers (right atrium RA, right ventricle RV, left atrium LA, left ventricle LV) and a left atrial appendage LAA 50.

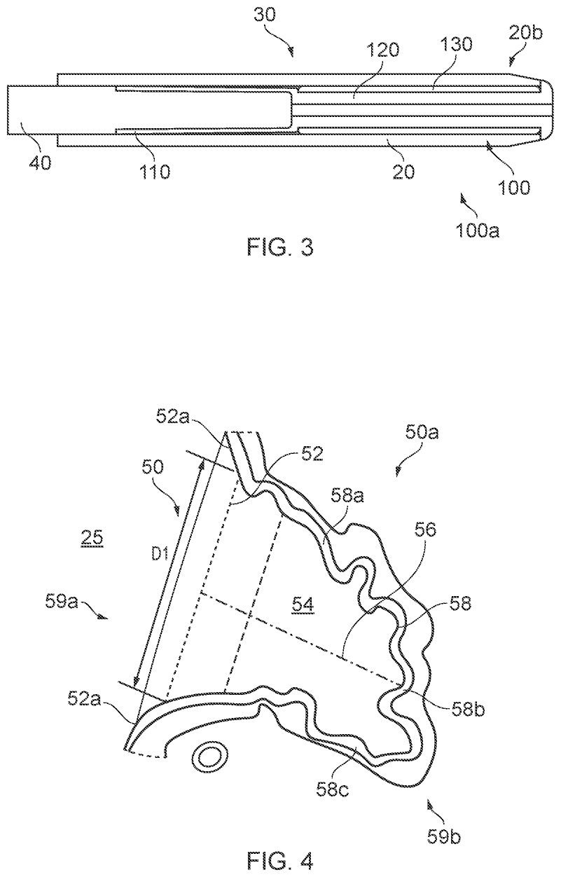

FIG. 2 illustrates a diagrammatic view of a patient undergoing an exemplary left atrial appendage occlusion procedure. As indicated by the enlarged circle area 2, an occlusion assembly 10 may be inserted into a vasculature 6 of a patient, including for example, into an artery 6a (e.g. a femoral artery) or vein (e.g., femoral vein, internal jugular vein). The occlusion assembly 10 includes a catheter 20 and an occlusion device 100.

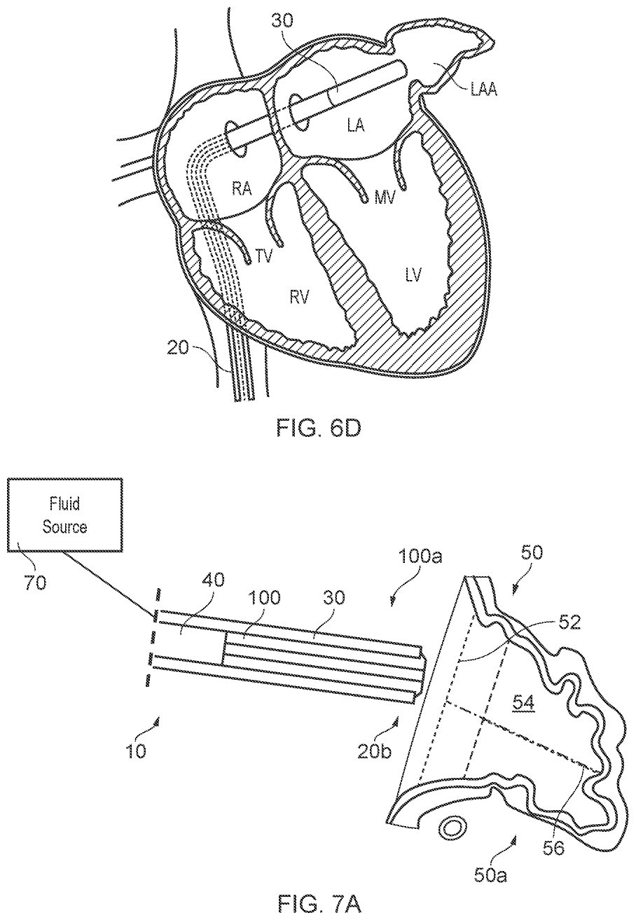

The catheter 20 is arranged to be positioned next to the left atrial appendage 50 within the heart 45. The catheter 20 has an elongate tubular body 22 and a central lumen 24 (shown more clearly in FIG. 5) that extends from a proximal end 20a of the catheter 20 to the distal end 20b of the catheter. As shown schematically in FIG. 2, the proximal end 20a of the catheter 20 is housed in a handle 200 of the occlusion device 10. The proximal end 20a of the catheter therefore remains outside of the patient's body while the distal portion 20b is advanced through the patients' vasculature 6 towards the left atrial appendage 50. A medical practitioner manipulates the position and orientation of the catheter 20 by using the handle 200. As shown, the handle 200 may also be connected to any number of systems known to those skilled in the art, generally indicated by system 250 in FIG. 2.

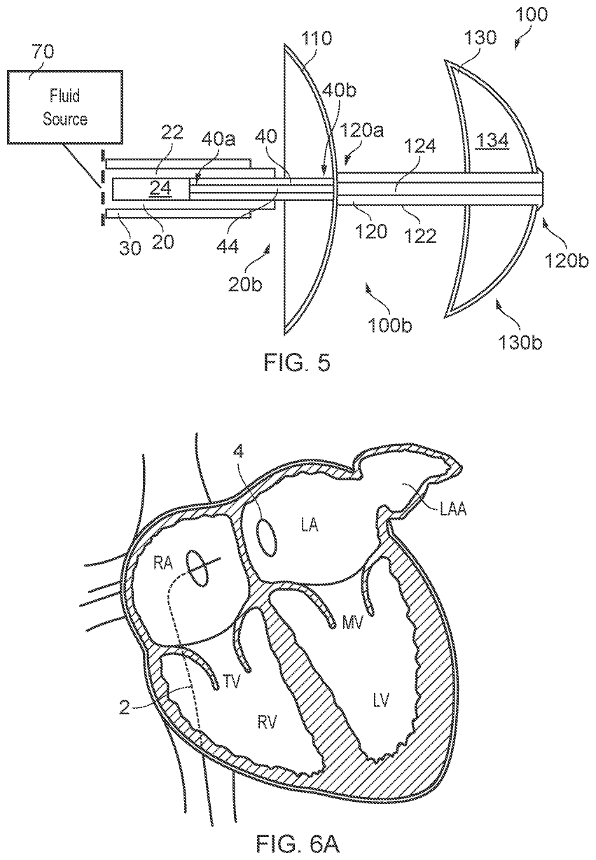

As shown in FIGS. 2 and 3, a capsule 30 is arranged at the distal end 20b of the catheter 20. FIG. 3 shows a cross-sectional view of the capsule 30 and the occlusion device 100. The capsule 30 contains the occlusion device 100 in a collapsed state 100a. The capsule 30 protects the occlusion device 100 as the distal end 20b of the catheter 20 navigates through the vasculature 6 of the patient. The capsule 30 may be securely connected to the distal end 20b of the catheter 20 or may be formed as part of the tubular body 22 of the catheter 20 at the distal end 20b.

The occlusion device 100 is selectively deployable from a collapsed state 100a to an expanded state 100b. A medical practitioner may deploy the occlusion device 100 by, for example, retracting the capsule 30 towards the proximal end 20a of the catheter 20, or by distally pushing the occlusion device 100 from the distal end 20b of the catheter 20. The capsule 30 may be retracted proximally only if the capsule 30 does not form part of the tubular body 22 of the catheter 20. In these examples, when the capsule 30 is withdrawn back into the catheter 20, the proximal portion 110 of the occlusion device 100 is released to seal area 52a surrounding the opening 52 of the left atrial appendage. In order to proximally retract the capsule 30, the capsule 30 must be first extended away from the tubular body 22 to provide clearance between the capsule 30 and the catheter 20. In some examples. the capsule 30 may be arranged to be withdrawn back into the catheter 20 when the capsule 30 is proximally retracted. The occlusion device 100 of the occlusion assembly 10 is arranged to occlude (or obstruct) a left atrial appendage 50.

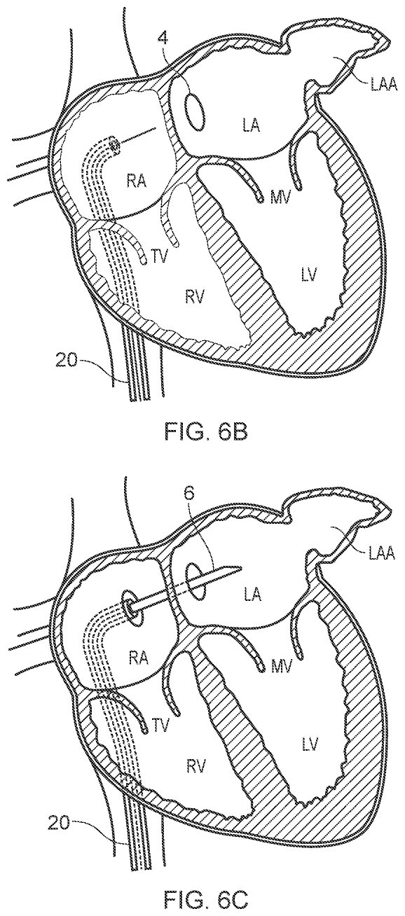

While the shape of left atrial appendage 50 will vary for among patients, the left atrial appendage 50 is shown in FIG. 4 as being generally bursiform (pouch) shaped and has an opening 52 at a proximal end 59a that extends to a distal end 59b. The opening 52 is generally circular in shape and has a diameter D1. As shown, the opening 52 generally corresponds to the widest part of the left atrial appendage 50.

The left atrial appendage 50 has an internal volume 54 that is in fluid communication with the left atrium LA. The internal volume 54 may contain a volume of blood from the heart 45. The internal volume 54 is partially enclosed by a wall 58. As shown, the wall 58 has an irregular shape that generally forms an ear-shaped structure. In this example, the left atrial appendage wall 58 is formed of a superior wall 58a, an inferior wall 58c and a rear wall 58b. The left atrial appendage wall 58 includes further anterior and posterior wall sections that are not shown in FIG. 4.

The left atrial appendage 50 may have any type of morphology. For example, the left atrial appendage 50 may have a depth greater than the opening diameter D1, a smaller than the opening diameter D1, have a chicken-wing shape, cactus shape or cauliflower shape.

The left atrial appendage has a depth 56 that extends from the opening 52 (at the proximal end 59a) to the rear wall 58b (at the distal end 59b). Generally, the depth 56 of the left atrial appendage 50 is greatest at the rear wall 58b. The left atrial appendage 50 is in a natural, pre-treated state 50a in FIG. 4.

An exemplary occlusion assembly 10 for occluding a left atrial appendage 50 is shown in FIG. 5. As shown, the occlusion device 100 is in an expanded state 100b with the capsule 30 in a proximally retracted position. A plunger 40 is at least partially housed in the central lumen 24 of the catheter 20. The plunger 40 has a generally tubular body 42 with a central lumen 44 that extends from a proximal end 40a to a distal end 40b. The distal end 40b of the plunger 40 is releasably connected to a proximal portion 110 of the occlusion device 100 (discussed further below). The distal end 40b of the plunger 40 may be releasably connected by any suitable mechanical connection, such as a breakaway connection. . . . The plunger 40 may be selectively detached from the occlusion device 100 by the medical practitioner. The occlusion device 100 is therefore arranged to remain in the left atrial appendage 50 after being detached from the catheter 20. The central lumen 44 of the plunger 40 is in fluid communication with the central lumen 24 of the catheter 20.

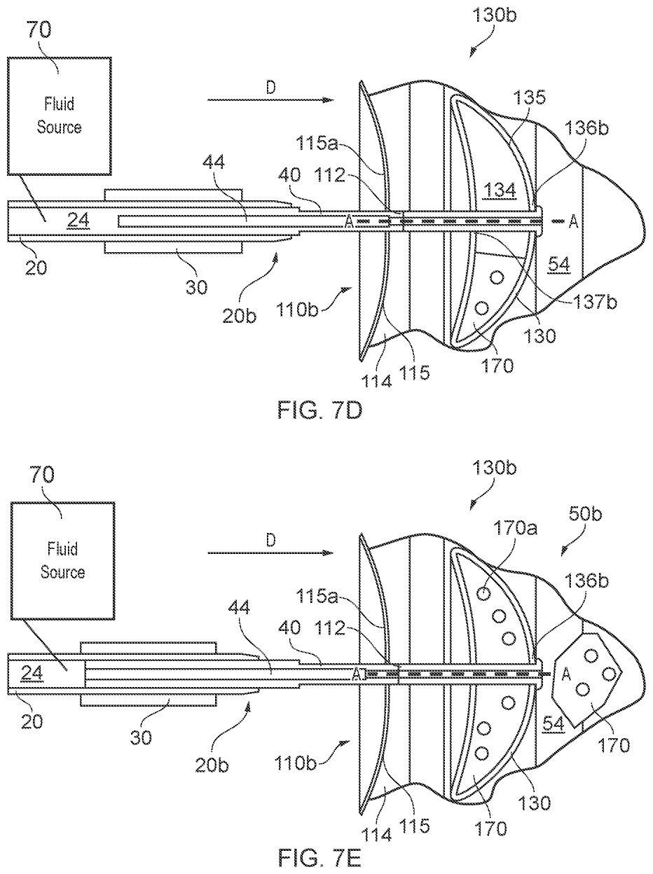

The occlusion device 100 includes a proximal portion 110, a stem 120 and a distal portion 130. As shown, the occlusion device 100 is arranged at the distal end 20b of the catheter 20 and is connected to the catheter 20 through the plunger 40. The occlusion device 100 is arranged to move from a collapsed state 100a (shown in FIGS. 3, 7A and 7B) to an inflated state 100b (shown in FIGS. 5, 7C and 7D). The occlusion device 100 is in a collapsed state 100a (i.e. not inflated) when the occlusion device 100 is housed inside the capsule 30. The occlusion device 100 is therefore in a collapsed state 100a as the catheter 20 is moved through the patient's vasculature 6 towards the left atrial appendage 50. As shown in FIG. 7A, the distal end 20b of the catheter is arranged to be positioned adjacent to the opening 52 of the left atrial appendage 50.

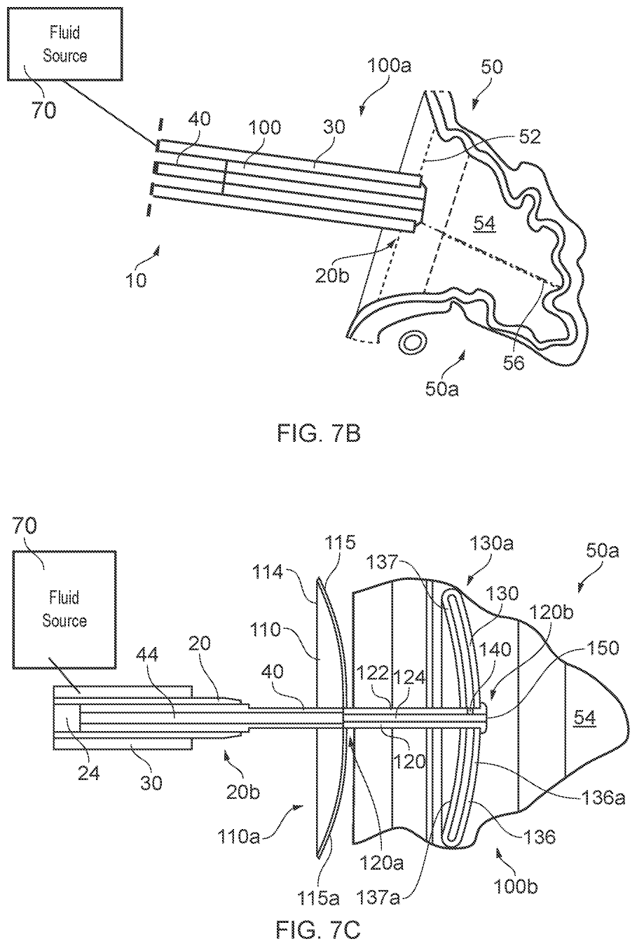

Referring to FIGS. 6A to 6D, a method for delivering and deploying an occlusion device to a left atrial appendage LAA will now be described. In an exemplary embodiment, a guidewire 2 is advanced after having been introduced into the vasculature via a percutaneous entry point and tracked through the vasculature into a left atrium LA of a heart. Intravascular access to the right atrium RA may be achieved via a percutaneous access site to femoral venous access up to the inferior venal cava, or other known access routes. Thereafter, a guidewire is advanced through the circulatory system, eventually arriving at the heart.

The guidewire 2 is directed into the right atrium RA (see FIG. 6A), traverses the right atrium and is made to traverse, with the aid of a pre-existing hole 4 (see FIG. 6B) or a transseptal needle 6 (see FIG. 6C) an atrial septum, thereby entering the left atrium LA. Once the guidewire 2 is positioned, the entry port and the atrial septum are dilated to permit entry of the catheter 20 into the left atrium LA towards the left atrial appendage LAA (see FIG. 6D).

There, the capsule 30 of the catheter 20 is positioned proximate the left atrial appendage LAA. Although described as a transfemoral antegrade approach for percutaneously accessing left atrium LA, the catheter 20 may be positioned within the desired area of the heart via different methods or routes. For example, and not by way of limitation, another possible path would be through the radial vein into the brachial vein, through the subclavian vein, through the superior vena cava into the right atrium, and then transeptally into the left atrium.

Yet another possible path would be through the femoral artery into the aorta (as shown in FIG. 2), through the aortic valve into the left ventricle, and then retrograde through the mitral valve into the left atrium. In another embodiment, the left ventricle LV may be accessed via a transapical approach, and the catheter 20 may be advanced through the left ventricle LV, the mitral valve, and into the left atrium LA adjacent the left atrial appendage LAA. In addition, although described with the use of a guidewire, in another embodiment hereof the catheter 20 may access the left atrium LA without the use of a guidewire.

As shown in FIGS. 7A and 7B, which show cross-sectional views of the capsule 30, the occlusion device 100 remains in a collapsed state 100a when the distal end 20b of the catheter 20 is positioned near the opening 52 of the left atrial appendage 50. Once the distal end 20b of the catheter 20 is adjacent to the opening 52, the distal end 20b of the catheter 20 is advanced through the opening 52 and into the volume 54 of the left atrial appendage 50, as shown in FIG. 7B.

When the capsule 30 is proximally retracted in FIG. 7C, the occlusion device 100 deploys into an expanded state 100b. Preferably, the capsule 30 is retracted after the proximal end 20b of the catheter 20 is advanced through the opening 52 of the left atrial appendage 50 to ensure that the expandable member 130 of the occlusion device 100 is positioned inside the volume 54 of the left atrial appendage 50.

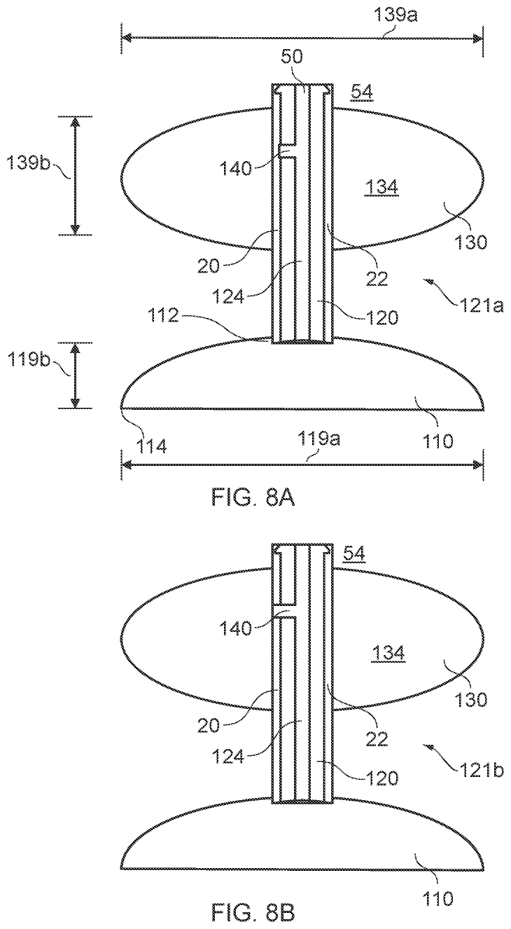

As shown, the occlusion device 100 includes a stem 120 with a substantially tubular body 122 and a central lumen 124 that extends from a proximal end 120a of the stem 120 to a distal end 120b. The proximal end 120a of the stem 120 is secured to the proximal portion 110 of the occlusion device 100. The proximal end 120a of the stem 120 may be secured to the proximal portion 110 through any suitable means, such as adhesive or any suitable mechanical engagement. The distal end 120b of the stem 120 is secured to an expandable member 130, which is in a collapsed configuration 130a in FIG. 7C. As shown more clearly in FIG. 8A, the tubular body 122 of the stem 120 passes through the expandable member 130 and is secured to an outer surface 136a of the distal face 136 of the expandable member 130. As shown, the distal face 136 is distal to the distal end 20b of the catheter 20.

The stem 120 provides fluid communication between the catheter 20 and the expandable member 130 and optionally, to the internal volume 54 of the left atrial appendage 50. The central lumen 124 of the stem 120 is in fluid communication with the central lumen 24 of the catheter 20 through the central lumen 44 of the plunger 40. The stem 120 is also in fluid communication with the expandable member 130 through an aperture 140 that extends perpendicularly from the central lumen 124 of the stem 120 (shown more clearly in FIG. 8A). In this example, only one aperture 140 extends from the central lumen 124 of the stem 120, but any number of apertures 140 may be used.

Optionally, the stem 120 may further comprise a distal aperture 150 that is arranged at the distal end 120b of the stem 120. The distal aperture 150 is in fluid communication with the internal volume 54 of the left atrial appendage 50, as shown more clearly in FIGS. 7C and 7D.

The expandable device 130 is arranged to move between a collapsed state 130a (shown in FIG. 7C) and an expanded state 130b (shown in FIG. 7D) after the occlusion device 100 is in a deployed state 100b. The expandable device 130 at least partially expands through the introduction of a fluid 170 through the aperture 140. The fluid 170 is introduced by a fluid source 70 attached to the catheter 20, and is delivered through the catheter 20, the plunger 40, through the stem 120 and into an internal volume 134 of the expandable member 130. In an expanded state 130b, the expandable member 130 at least partially occludes the internal volume 54 of the left atrial appendage 50.

The expandable member 130 is positioned at the distal end 120b of the stem 120 and is arranged to be positioned inside the left atrial appendage 50. The expandable member 130 has a width 139a (shown in FIG. 8A) that is arranged to extend across the width of the left atrial appendage 50 (i.e. from walls 58a to 58c) and a height 139b that is arranged to extend along the depth 56 of the left atrial appendage 50 (i.e. from the opening 52 to the rear wall 58b).

The expandable member 130 can be substantially asymmetrical. Preferably, the width 139a of the expandable member 130 is larger than the height 139b, so that the width 139a of the expandable member 130 can substantially extend across the width of the left atrial appendage 50. The width 139a of the expandable member 130 is substantially oval or elliptical shaped so that the expandable member 130 may generally conforms to the internal profile of the left atrial appendage 50. By having a larger width 139a, the expandable member may be easily positioned and seated inside the left atrial appendage 50 with less risk of the device migrating back out of the opening 52 and back into the heart 45. This also increases the surface area of the expandable device 130 that contacts against the left atrial appendage walls 58a-c. This arrangement helps prevent the occlusion device 100 from migrating back out of the left atrial appendage 50.

As shown in FIG. 7C, the expandable member 130 is arranged to be positioned and substantially extend across the widest part of the left atrial appendage 50 that is distal from the opening 52. This increases the friction between the expandable member 130 and the walls 58a-c of the left atrial appendage 50. This ensures that when the occlusion device 100 is in an expanded state 100b, the expandable member 130, although in a collapsed state 130a, will be less likely to migrate back through the opening 52 of the left atrial appendage 50. This also ensures that the expandable member 130 will occlude a large volume of the internal volume 54 of the left atrial appendage 50 when the expandable member 130 is at least partially expanded into an expanded state 130b.

The expandable member 130 has an expandable frame 135 with a distal face 136, a proximal face 137 and an internal volume 134. The expandable frame 135 may be formed of any suitable bioinert expandable material but is preferably formed of nitinol mesh. The nitinol mesh is not only compliant (and therefore may deform easily if the expandable frame 135 contacts any of the walls 58a-c of the left atrial appendage 50) but also expands in response to higher temperatures in the body. The expandable frame 135 therefore easily expand in the left atrial appendage 50 after the capsule 30 is retracted.

The distal face 136 of the expandable member 130 has a distal outer surface 136a and the proximal face 137 of the expandable member 130 has a proximal outer surface 137a. Preferably, the distal outer surface 136a is generally dome-shaped with an apex 136b. As shown in FIG. 7D, the apex 136b of the distal outer surface 136 is distal from the distal end 20b of the catheter 20. Similarly, the proximal outer surface 137a is substantially domed with an apex 137b. The stem 120 is arranged to extend through the expandable member 130 through both apexes 136b, 137b.

The proximal end 120a of the stem 120 is connected to a proximal portion 110. As shown in FIG. 7C, the proximal portion 110 is generally dome shaped, with a rim 114 that extends around a cup portion 115. The cup portion 115 has an outer surface 115a and has an apex 112. The rim 114 of the proximal portion 110, in a collapsed state, is proximal to the distal end 20b of the catheter 20, while the apex 112 of the proximal portion 110 is distal to the distal end 20b of the catheter 20. The cup portion 115 is substantially circular to conform with the opening 52 of the left atrial appendage 50.

The proximal portion 110 is arranged to be brought into sealing engagement against the opening 52 of the left atrial appendage (discussed further below). The proximal portion 110 is pre-engaged configuration 110a in FIG. 7C, while FIG. 7D shows the proximal portion 110 in an engaged configuration 110b (i.e. sealing the opening 52 of the left atrial appendage 50). The proximal portion 110 has a width 119a that is arranged to extend across the width of the opening 52 of the left atrial appendage 50 and a height 119b that is arranged to extend into the left atrial appendage 50 through the opening 52. The outer surface 115a of the proximal portion 110 is arranged to contact against an area 52a surrounding the opening 52 to seal the opening 50. Preferably, the rim 114 of the proximal portion 110 is larger than the opening 52 so that the outer surface 115a of the proximal portion 110 contacts most of the area 52a surrounding the opening 52.

In this example, the proximal portion 110 is substantially circular with an apex 112. The substantially dome shape of the proximal portion 110 increases the sealing effect against the opening 52 of the left atrial appendage 50 as the proximal portion 110 provides a suctioning effect against the opening 52. However, in other examples, the proximal portion 110 may be oval, elliptical or any other suitable shape to conform to the openings 52a of the left atrial appendage 50. The appropriate shape and size of the proximal portion 110 may be determined by the medical practitioner using any suitable technique, such as injecting dye or radio imaging techniques. The size of the proximal portion 110 and the expandable member 130 may therefore be adjusted depending on the morphology of the left atrial appendage 50 of the patient. The proximal portion 110 seals the opening 52 of the left atrial appendage 50 to prevent any material, such as blood or clots from exiting or entering the left atrial appendage 50. The proximal portion 110 also acts as a plug that helps secure the occlusion device 100 in the left atrial appendage 50 to prevent migration of the occlusion device 100.

The proximal portion 110 is arranged to contact against the tissue forming the area 52a surrounding the opening 52 of the left atrial appendage 50 for extended periods of time. Therefore, at least the outer surface 115a of the proximal portion is formed from a bioinert polymer, such as PDMS. This is to ensure that the proximal portion 110 does not cause an inflammatory response in the area 52a surrounding the opening 52. The proximal portion 110 may also be partially coated with a material coating such as PLGA (poly(lactic-co-glycolic acid)). The outer surface 115a of the cup portion 115 may be treated with oxygen plasma to increase surface adhesion to the PLGA material. The PLGA material coating may also be used to help secure adhesion of the cup portion 115 to the area 52a surrounding the opening 52 of the left atrial appendage 50.

The proximal portion 110 is arranged to be brought into sealing engagement with the opening 52 of the left atrial appendage 50 as the expandable member 130 is expanded. As described above, the expandable member 130 is arranged to at least partially expand by receiving fluid 170 from a fluid source 70 through the aperture 140 of the stem 120. The fluid source 70 is in fluid communication with the central lumen 24 of the catheter 20. Therefore, the fluid source 70 is operable to deliver fluid 170 through the plunger 40 and the stem 120 to the internal volume 134 of the expandable member 130.

The expandable member 130 is arranged to expand as the fluid 170 enters into the internal volume 134 of the expandable member 130. The fluid 170 is preferably any suitable inert fluid, such as biogel, saline or contrast media. The fluid 170 is used to expand the expandable member 130 to occlude the left atrial appendage 50 and also helps maintain the expandable member 130 in the expanded state 130b. The fluid 170 may include radiopaque markers 170a so that the injection and delivery of fluid into the expandable member 130 may be observed by the medical practitioner through additional imaging techniques, such as fluoroscopy. The radiopaque markers 170a therefore help the medical practitioner to ensure that the occlusion device 100 is positioned correctly in the left atrial appendage 50. The radiopaque markers 170a also help guide the medical practitioner in providing the correct amount of fluid 170 into the expandable device 130 depending on the size of the left atrial appendage 50, which can vary depending on the morphology of the patient.

As the expandable member 130 is asymmetrical, the expandable member 130 does not evenly expand in the left atrial appendage 50, i.e. the distal face 136 and the proximal face 137 of the expandable frame 135 do not displace equally or at the same rate. Generally, the distal face 136 of the expandable member 130 is displaced further towards the distal end 59b of the left atrial appendage 50 than the proximal face 137. As the distal end 120b of the stem 120 is secured to the distal outer surface 136a of the expandable member 130, the expandable member 130 displaces the stem 120 as the expandable member 130 is filled with fluid 170.

As shown in FIG. 7D, the stem 120 is displaced in a direction D towards the distal end 59b of the left atrial appendage 50. The proximal portion 110 of the occlusion device is also displaced in a direction D as the expandable member 130 expands because the proximal end 120a of the stem 120 is secured to the proximal portion 110. The proximal portion 110 is therefore brought into sealing engagement against the opening 52 of the left atrial appendage 50 as the expandable member 130 is filled with fluid 170. Expanding the expandable member 130 occludes the internal volume 54 of the left atrial appendage 50 and simultaneously seals the opening 52 of the left atrial appendage 50 with the proximal portion 110. The occlusion assembly 10 therefore allows the medical practitioner to occlude and seal the left atrial appendage 50 in one procedural step without additional devices or equipment.

The occlusion device 100 is arranged to be left implanted in the left atrial appendage 50 after the expandable member 130 has been sufficiently expanded and the proximal portion 110 is brought into scaling engagement against the opening 50 of the left atrial appendage 50. The plunger 40 is releasably connected to the proximal portion 110 of the occlusion device 110 by any suitable means, such as a mechanical snap feature or a threaded arrangement. The plunger 40 may be detached by the medical practitioner by mechanically snapping the plunger 40 from the proximal portion 110, or by disengaging the threaded arrangement. After the distal end 40b of the plunger 40 is detached, the plunger 40 is retracted proximally into the distal end 20b of the catheter 20 before the catheter 20 is withdrawn from the patient. The occlusion device 100 remains in the left atrial appendage 50. FIG. 7E shows the left atrial appendage 50 in an occluded state 50b.

The stem 120 may have a further distal aperture 150 at the distal end 120b of stem 120. As shown in FIG. 7C, the distal aperture 150 is in fluid communication with the internal volume 54 of the left atrial appendage 50. After the expandable member 130 has been expanded into an expanded state 130b, the fluid source 70 may be used to deliver fluid 170 into the remaining internal volume 54 of the left atrial appendage 50. The fluid 70 may be delivered to occlude the remaining volume of the left atrial appendage 50 or to help secure the occlusion device 100 in place and prevent migration of the occlusion device 100.

The stem 120 has at least one aperture 140 in fluid communication with the internal volume 134 of the expandable member 130, and at least one aperture 150 at the distal end of the stem 120. However, the stem 120 may have any number or arrangement of apertures 140, 150 to insert fluid into the expandable member 130 and the internal volume 54 of the left atrial appendage respectively. FIGS. 8A and 8B show an exemplary cross-sectional view of the stem 120 that is moveable between a first position 121a and a second position 121b. FIGS. 8A and 8B show horizontal cross sections along line A-A of the stem 120 shown in FIG. 7C. In FIG. 8A, one aperture 140 extends from the central lumen 124 of the stem 120 and is in fluid communication with the internal volume 134 of the expandable member 130. Similarly, as shown in FIG. 7C, one distal aperture 150 is at the distal end 120b of the stem 120 and is in fluid communication with an internal volume 54 of the atrial appendage 50.

In some examples, the stem 120 may include a threaded engagement (not shown) that is operable by the medical practitioner to control the apertures 140, 150. The threaded engagement may be used to selectively occlude the aperture 140 or the distal aperture 150. The threaded engagement is operable by the medical practitioner so that the stem 120 is moveable between a first position 121a and a second position 121b to selectively occlude the aperture 140 or the distal aperture 150.

FIG. 8A shows the stem 120 in the first position 121a. In this position, the aperture 140 is occluded by the threaded engagement (not shown) and no longer in fluid communication with the internal volume 134 of the expandable member 130. The distal aperture 150 remains in fluid communication with the left atrial appendage 50. Therefore, when fluid 170 is introduced through the central lumen 124 of the stem 120, the fluid 170 is delivered through the distal aperture 150 and into the internal volume 54 of the left atrial appendage 50.

FIG. 8B shows the stem in the second position 121b. In the second position 121b, the aperture 140 remains in fluid communication with the internal volume 134 of the expandable member 130 while the distal aperture 150 is occluded by the threaded engagement (not shown) and no longer in fluid communication with the internal volume 54 of the left atrial appendage 50. Therefore, when fluid 170 is introduced through the central lumen 124 of the stem 120, the fluid 170 is delivered through the aperture 140 and into the internal volume 134 of the expandable member 130.

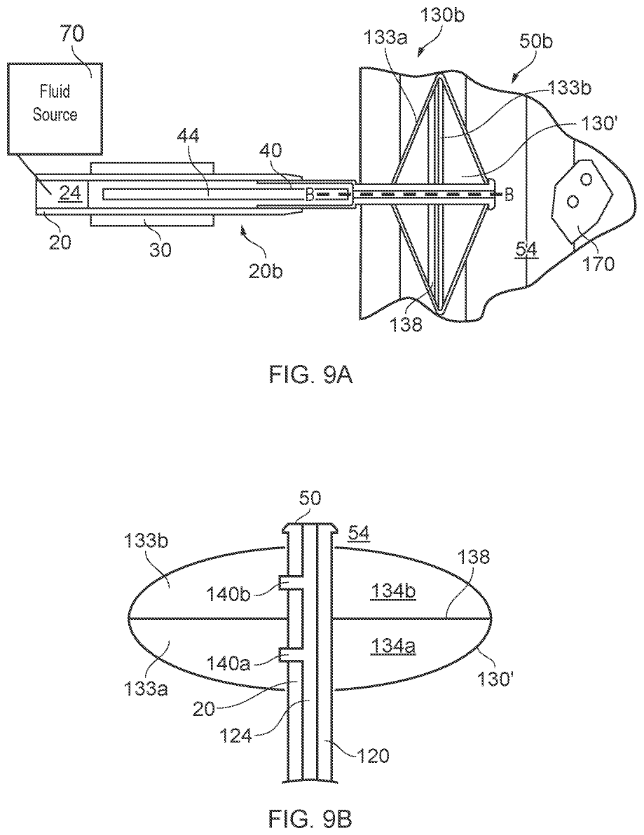

FIGS. 9A and 9B show another exemplary embodiment of the occlusion device 100. The proximal portion 110 is omitted from these figures for ease of clarity. In this embodiment, the expandable member 130′ has a first internal chamber 133a and a second internal chamber 133b that are separated by a wall 138. The first chamber 133a has an internal volume 134a and the second chamber 133b has an internal volume 134b. In this example, the wall 138 extends along the width 139 of the expandable member 130, but it will be understood that the chambers 133a, 133b may be formed by placing the wall 138 along any orientation in the expandable member 130. Furthermore, any number of chambers 133a, 133b may be formed inside the expandable member 130 for better control of the sealing or filling of the occlusion device 100

FIG. 9B is a cross-sectional view of the stem 120 and the expandable member 130 along line B-B shown in FIG. 9A. In this example, the stem 120 has a first aperture 140a in fluid communication with the first internal chamber 133a and a second aperture 140b in fluid communication with the second internal chamber 133b. In this arrangement, each chamber is arranged to selectively expand with the introduction of fluid 170 into each internal volume 134a, 134b respectively. Similar to the stem 120 shown in FIGS. 8A and 8B, the stem 120 may include a dual step threaded engagement that is operable to occlude each aperture 140a, 140b independently. In this configuration, the threaded engagement may be activated between a first position and a second position (not shown) to selectively occlude each aperture 140a, 140b respectively. This enables the medical practitioner to selectively expand each chamber 134a, 134b of the expandable member 130. The expandable member 130′ may therefore be selectively expanded depending on the shape of the internal volume 54 of the left atrial appendage 50. The expandable member 130′ may there be adapted to occlude different shapes or sizes of the left atrial appendage 50.

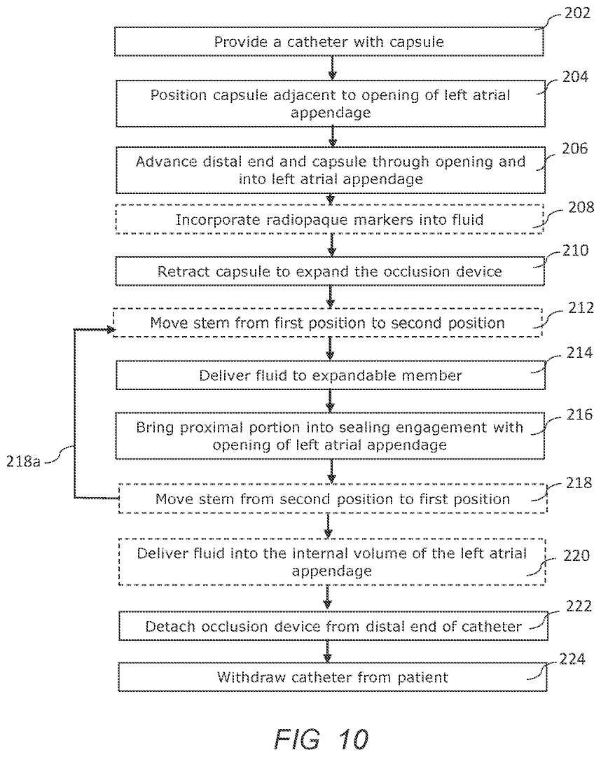

An exemplary occlusion assembly 10 including an exemplary occlusion device 100 for occluding a left atrial appendage 50 is described above. An exemplary method for occluding the left atrial appendage is described below in relation to FIG. 10.

At step 202, an occlusion assembly 10 is provided with a catheter 20. The occlusion device 100 is loaded into a capsule 30 in a collapsed state 100b and is arranged on the distal end 20b of the catheter 20. The distal end 20b of the catheter 20 is positioned adjacent to the opening 52 of the left atrial appendage in step 204. As shown in FIG. 7B, the distal end 20b of the catheter 20 is advanced through the opening 52 towards the distal end 59b of the left atrial appendage 50 in step 206. As shown in FIG. 7D, the distal end of the catheter 20b is positioned inside the volume 54 of the left atrial appendage 50.

A fluid source 70 is connected to the catheter 20 and is arranged to deliver biogel 170 through the catheter 20 and into the occlusion device 100. Optionally, at step 208, radiopaque markers 170a may be incorporated into the biogel 170 before introducing the biogel 170 into the occlusion device 100.

The capsule 30 is then retracted proximally to move the occlusion device 100 from a collapsed state 100a to an expanded state 100b at step 210, as shown in FIG. 7C. In the expanded state 100b, the expandable member 130 is positioned inside the left atrial appendage 50.

Optionally, if the stem 120 has a plurality of apertures 140a, 140b and the stem 120 is arranged to be moveable between a first position 121a and a second position 121b, the stem is moved by the medical practitioner at step 121 to be in a second position 121b. In the second position 121b, at least one aperture 140 is in fluid communication with the internal volume 134 of the expandable member 130.

At step 214, biogel is delivered to the expandable member 130 with the fluid source 70. The expandable member 130 is at least partially expanded at step 214. The biogel 170 is delivered through the central lumen 24 of the catheter 20, through the central lumen 44 of the plunger 40, through the central stem 124 of the stem 120 and through at least one aperture 140 into the expandable member 130. The expandable member 130 expands to occlude the internal volume 54 of the left atrial appendage 50.

As the expandable member 130 expands at step 214, the proximal portion 110 of the occlusion device 100 is brought into sealing engagement with the opening 52 of the left atrial appendage 50 at step 216. The proximal portion 110 therefore seals the opening 52 of the left atrial appendage 50 from the left atrium LA.

Optionally, at step 218a, the stem 120 may be moved back into the first position 121b to occlude the aperture 140 before moving on to step 220. If the expandable member 130′ has multiple apertures 140a, 140b, then the medical practitioner may return to step 212 to move the stem 120 back into a first position 121a to occlude the first aperture 140a so that the second chamber 133a may receive fluid through the second aperture 140b in step 214.

After the aperture 140 is in the first position 121a and is occluded, the medical practitioner may introduce biogel 170 into the internal volume 54 of the left atrial appendage 50 at step 220 through the distal aperture 150.

After the biogel 170 has been delivered inside to the expandable member 130 and optionally inside the left atrial appendage 50, the occlusion device 100 is detached from the distal end 20b of the catheter 20 at step 222. More specifically, the distal end 40b of the plunger 40 is detached from the proximal portion 110 by the use of a threaded engagement. Preferably, the distal end 40b of the plunger 40 is detached from the occlusion device 100 after the proximal portion 100 has created a seal against the opening 50 of the left atrial appendage. The distal end 20b of the catheter 20 (with the plunger 40) is then withdrawn from the patient at step 224.

With regard to the terms “distal” and “proximal” within this description, unless otherwise specified, the terms can reference a relative position of the portions of the delivery catheter system with reference to a medical practitioner and/or a location in the vasculature or heart. For example, “proximal” can refer to a position closer to the medical practitioner of the device or an incision into the vasculature, and “distal” can refer to a position that is more distant from the medical practitioner of the device or further from the incision along the vasculature (e.g., the end of the catheter).

Although the teachings have been described above with reference to one or more preferred embodiments, it will be appreciated that various changes or modifications may be made without departing from the scope as defined in the appended claims.

Where the word ‘or’ appears this is to be construed to mean ‘and/or’ such that items referred to are not necessarily mutually exclusive and may be used in any appropriate combination.

Although the invention has been described above with reference to one or more preferred embodiments, it will be appreciated that various changes or modifications may be made without departing from the scope of the invention as defined in the appended claims.

1. An occlusion device for occluding a left atrial appendage, the device comprising:
a stem with an elongate tubular body and a central lumen that extends from a proximal end to a distal end,
a proximal arrange at the end of the stem;
wherein the proximal portion is arranged to engage with an opening of the left atrial appendage,
an expandable member arranged at the distal end of the stem;
wherein the central lumen of the stem is in fluid communication with the expandable member and wherein the expandable member is arranged to be positioned inside a left atrial appendage; and
wherein the expandable member is operable to at least partially expand and displace the proximal portion in a direction towards the expandable member.

2. The occlusion device of claim 1, wherein the expandable member is arranged to receive fluid through the central lumen of the stem so as to at least partially expand within the left atrial appendage.

3. The occlusion device of claim 1, wherein the expandable member is asymmetric, having a width greater than its height.

4. The occlusion device of claim 3, wherein the width of the expandable member is substantially oval or elliptical shaped.

5. The occlusion device of claim 1, wherein the expandable member comprises a nitinol mesh.

6. The occlusion device of claim 1, wherein the distal end of the stem extends through the expandable member and is secured to an outer surface at a distal end of the expandable member.

7. The occlusion device of claim 1, wherein the stem is in fluid communication with the expandable member through at least one aperture extending from the central lumen of the stem.

8. The occlusion device of claim 7, wherein the aperture extends perpendicularly from the central lumen.

9. The occlusion device of claim 8, wherein the stem is arranged to be positioned in a distal end of a catheter so that the distal end of the catheter extends around the stem, wherein the stem is moveable between a first position and a second position, wherein in the first position, the at least one aperture is occluded and no longer in fluid communication with the expandable member, and
in a second position, the at least one aperture is in fluid communication with the expandable member.

10. The occlusion device of claim 7, wherein the stem further comprises at least one aperture at the distal end of the stem, wherein the at least one aperture is arranged to be in fluid communication with the left atrial appendage.

11. The occlusion device of claim 7, wherein the expandable member comprises at least two chambers.

12. The occlusion device of claim 11, wherein the stem is in fluid communication with each chamber through at least one respective aperture so that each chamber is arranged to be expanded separately.

13. The occlusion device of claim 1, wherein the proximal portion is dome shaped and an apex of the dome is proximal to the expandable member.

14. The occlusion device of claim 1, wherein the proximal portion comprises a bioinert polymer.

15. The occlusion device of claim 1, wherein the proximal portion is at least partially coated with a material coating.

16. An occlusion assembly for occluding a left atrial appendage, the assembly comprising:
a catheter with an elongate tubular body and a central lumen that extends from a proximal end to a distal end of the catheter, the distal end arranged to be positioned at an opening of the left atrial appendage;
an occlusion device comprising a stem with an elongate tubular body and a central lumen that extends from a proximal end to a distal end,
a proximal portion arranged at the proximal end of the stem;
wherein the proximal portion is arranged to engage with an opening of the left atrial appendage,
an expandable member arranged at the distal end of the stem;
wherein the central lumen of the stem is in fluid communication with the expandable member and the expandable member is arranged to be positioned inside a left atrial appendage; and
wherein the expandable member is operable to at least partially expand and displace the proximal portion in a direction towards the expandable member, wherein the occlusion device is releasably connected to the distal end of the catheter.

17. The occlusion assembly according to claim 16, wherein the occlusion assembly further comprises a fluid source coupled to the catheter, wherein the fluid source is operable to deliver fluid to the expandable member.

18. The occlusion device of claim 17, wherein when the stem of the occlusion device further comprises at least one aperture at the distal end of the stem and wherein the at least one aperture is arranged to be in fluid communication with the left atrial appendage, and wherein the fluid source is further operable to deliver fluid into the left atrial appendage.

19. The occlusion assembly according to claim 17, wherein the occlusion assembly further comprises a plunger with a proximal end and a distal end,
wherein the proximal end of the plunger is housed in the distal end of the catheter and the distal end of the plunger is releasably connected to the proximal portion of the occlusion device, so that the occlusion device is releasably connected to the catheter through the plunger.

20. The occlusion assembly according to claim 17, wherein the fluid is one of: biogel, saline or contrast media.
